(19) United States
(12) Patent Application Publication
(10) Pub. No.: US 2003/0049304 A1
(43) Pub. Date: Mar. 13, 2003

(54) Quiescently frozen ice products

(76) Inventors: Jitendra Krishan Somani, Toronto (CA); Luigi Levi, Richmond Hill (CA); Joseph Lee, Toronto (CA); Michael Paul Ramsay, Ajax (CA)

Correspondence Address: Darryl C. Little, Attorney for Applicant, Warner Lambert Company, 201 Tabor Road, Morris Plains, NJ, 07950, US

(21) Appl. No.: 10/187,157

(22) Filed: Jun. 28, 2002

Related U.S. Application Data
(60) Provisional application No. 60/315,938, filed on Aug. 29, 2001.

(51) Int. Cl. A61K 9/14; A61K 47/00

(52) U.S. Cl. 424/439; 424/488

(57) ABSTRACT

The present invention relates to quiescently frozen ice products and there production. More specifically, the present invention relates to frozen ice products containing salts or esters of alginic acid for improved freezability.

This application claims the benefit of U.S. Provisional Patent Application No. 60/315,938, filed on Aug. 29, 2001, the entirety of which is hereby incorporated by reference as if fully set forth herein.

### FIELD OF THE INVENTION

The present invention relates to quiescently frozen ice products and there production. More specifically, the present invention relates to frozen ice products containing salts or esters of alginic acid for improved freezability.

### BACKGROUND OF THE INVENTION

Frozen ice products have been apart of our culture for quite some time. From the “snowball” to the more familiar italian ices, freezer pops and frozen fruit juices, these frozen novelties have long been a popular item with consumers. Frozen products of this type are, typically, carried on sticks, in plastic tubes or in wax coated paper containers and eaten directly from the freezer. No utensils or plates are necessary. And, recently, these products have enjoyed even greater commercial success due in part to the public's heightened awareness of such health concern as calories, cholesterol, fat and chemical-additives.

The use of frozen ice products to deliver drug or nutritional agents has also gained in popularity over the last few years. Due to the nature of the ingredients involved, various safeguards are being considered which restrict child access to such products.

One attempt at providing such a safeguard considers the possibility of storing the active containing ice products in child resistant box or box-like containers or cartons. However, at solid contents greater than 8% of the ice product formulation and at temperatures above −20° C., such containers tend to inhibit the freezing of the ice product composition. Either the ice products fail to freeze properly or remain liquid. Hence, a need exists for frozen product formulations which freeze solidly and uniformly in package containers.

Present inventors have found that by incorporating salts or esters of alginic acid the freezability (i.e., ability to freeze) of frozen ice products in containers or cartons is improved such that such containers remain a viable option for restricting access to medicated frozen ice products.

Accordingly an aspect of the present invention is to provide improved frozen ice products.

Another aspect of the present invention is to provide frozen ice product formulations which facilitate freezing of the frozen ice product at temperatures of from −5° C. to −20°.

Still another aspect of the present invention is to provide frozen ice product formulations of specific solids content comprising pharmaceutically acceptable salts of alginic acid to facilitate freezing of the frozen ice product at temperatures of from −5° C. to −20°.

These and other aspects of the present invention are described in detail below.

### SUMMARY OF THE INVENTION

The present invention in one of its aspects relates to frozen compositions, comprising:

a.) an amount of a freezing facilitator consisting essentially of a pharmaceutically acceptable salt or ester of alginic acid such that the viscosity of the composition prior to freezing is no more than 300 cps;

b.) optionally, a safe and effective amount of pharmaceutical active;

c.) optionally, a flavor;

d.) optionally, a sweetener; and

e.) water

wherein the frozen composition contains a solids content greater than 8% and less than 30% (or about 30%), preferably less than 25% (or about 25%) and most preferably a solids content of from 9% (or about 9%) to 20% (or about 20%) and wherein the frozen compositions are frozen quiescently within the container at a temperature range of from −5° C. (or about −5° C.) to about −20° C. (or about −20° C.).

All percentages and ratios used herein are by weight unless otherwise specified. Additionally, all measurements are made at 25° C. unless otherwise specified.

The compositions of the present invention can comprise, consist essentially of, or consist of, the essential as well as optional ingredients and components described herein. As used herein, “consisting essentially of” means that the composition or component may include additional ingredients, but only if the additional ingredients do not materially alter the basic and novel characteristics of the claimed compositions or methods.

The phrase “safe and effective amount”, as used herein, is defined as an amount of a substance sufficient to provide the desired benefit without undue adverse side effects, such as toxicity, irritation, or allergic response, commensurate with a reasonable benefit/risk ratio when used in the manner of this invention.

The term “pharmaceutically acceptable salts”, as used herein, means salts of alginic acid which have the same general properties as the compounds from which they are derived, and which are acceptable from a toxicity viewpoint.

The term “frozen,” as used herein, means that the product is solidified under freezing conditions, with or without air incorporation to a harden, firm consistency.

The term “quiescently frozen” as used herein means that the product has been statically frozen, that is, frozen without the accompaniment of stirring or agitation.

### DETAILED DESCRIPTION OF THE INVENTION

The essential as well as optional components of the capsules of the present invention are described in the following paragraphs.

### Essential Components

Salts of Alginic Acid

An essential component of the present invention is a pharmaceutically acceptable salt of alginic acid. Alginic acid is a polysaccharide made up of D-mannuronic acid and L-guluronic acid residues. It is produced from brown seaweed (Phaeophycae) through a variety of extraction processes. Depending on the particular species of seaweed and the method used in extraction, alginates of various molecular weights can be prepared.

It has now been found that pharmaceutically acceptable salts of alginic acid, particularly water-soluble salts of alginic acid facilitate the freezing of frozen ice products. The water-soluble alginates used in the present invention are generally the alkali metal salts of alginic acid such as potassium or sodium alginate. Also useful herein are esters of alginic acid such as propylene glycol esters of alginic acid (e.g., 1,2-propylene glycol esters). Preferred for use herein is sodium alginate. Useful grades of alginic acid include the Manugel series of alginates such as Manugel DMB, supplied by ISP Technologies, Proctin series supplied by FMC Biopolymer, the Protanal series of alginates (available with varying specifications of viscosity) such as Protanal LF 5/60, Protanal LF 10/60 and Protanal LF200 (M, DL or S), supplied by FMC Biopolymer as well as the Protanal Ester series supplied by FMC Biopolymer and the Kelcoloid series of alginate esters such as Kelcoloid LVF (propane-1,2-diol alginate) supplied by ISP Technologies (ISP Alginates). Preferred for use herein are the Protanal LF200 grades M, DL and S.

The esters and/or salts of alginic acid are incorporated into the frozen compositions of the present invention at amounts such that the viscosity of the frozen compositions is no more than 300 cps (or about 300 cps), preferably from 25 cps (or about 25 cps) to 200 cps (or about 200 cps), more preferably from 50 cps (or about 50 cps) to 150 cps (or about 150 cps) and most preferably from 75 cps (or about 75 cps) to 125 cps (or about 125 cps) as measured at 25° C. using a Brookfield viscometer Model LVT with a #1 spindle at 30 rpm.

It should be understood that different grades of salts or esters of alginic acid as well as mixtures thereof can be incorporated at varying amounts by persons skilled in the art to achieve the above-mentioned viscosities.

Water

Water is also present in the frozen ice products of the present invention. Water comprises from about 50% to about 95%, preferably from about 70% to about 90% of the frozen ice products described herein. These amounts of water include the free water which is added, plus that amount which is introduced with other materials such as with corn syrup. The water, used in the present invention should preferably be deionized, distilled, free of organic impurities and bacteria and substantially free of metal ions.

### Optional Components

The frozen ice products of the present invention can, optionally, include one or more pharmaceutically active ingredients. The active ingredient may be at least one member selected from the group consisting of nourishing and health-promoting agents, antipyretic-analgesic-inflammatory agents (such as aspirin, NSAIDS and acetaminophen), antipsychotic drugs, antianxiety drugs, antidepressants, hypnotic-sedative agents, spasmolytics, gastrointestinal function conditioning agents, antacids, antitussive-expectorants (such as dextromethorphan and guaifenesin), dental buccal drugs, antihistamines, cardiotonics, antiarrhythmic drugs, diuretics, antihypertensive drugs, vasoconstrictors, coronary vasodilators, peripheral vasodilators, cholagogues, antibiotics, chemotherapeutic drugs, antidiabetic agents, drugs for osteoporosis, skeletal muscle relaxants and mixtures thereof. A more detailed list of pharmaceutical actives suitable for use in the present invention can be found in U.S. Pat. No. 5,720,974, issued Feb. 24, 1998, herein incorporated by reference in its entirety.

Demulcents can also be used in the frozen compositions of the present invention. Demulcents are usually water-soluble polymers which are applied topically to protect, lubricate and coat the mucous membrane surfaces of the throat to relieve irritation. Suitable demulcents include demulcents such as elm bark, glycerin, pectin and mixtures thereof.

A food grade acid is generally employed in the frozen ice products of the present invention to improve taste. The food grade acid may be chosen from, but not limited to, the group consisting of adipic, fumaric, citric, tartaric or any other food grade acid known to the art. A combination of food acids from the above group is suitable for the invention. Generally, food grade acid is added at a level of from about 0.2% to about 0.7% by weight of the frozen confection, and typically from about 0.3% to about 0.6% by weight. A buffer, such as trisodium citrate, disodium phosphate, potassium phosphate, sodium tartrate, etc., may additionally be included for pH process control purposes. The pH of the confection generally ranges from 3.0-5.0 and is not considered critical to the functionality of the invention, but rather is important organoleptically.

A sweetener may also be incorporated into the frozen ice products of the present invention. Carbohydrate sweeteners, such as sucrose, dextrose, fructose, glucose, etc., are most often employed. Carbohydrate sweeteners are generally included at a level of about 5 to 50% by weight of the frozen confection, and preferably about 5 to 20% by weight. Carbohydrate sweeteners so employed additionally act to depress the freezing point of the frozen confection such that the frozen confection is ready-to-eat at freezer temperatures. Optionally, freezing point depressants such as salts or polyhydric alcohols may be employed to further depress the frozen confection freezing point. However, it is preferred that said carbohydrate sweeteners not be supplemented with specific freezing point depressants.

In a preferred embodiment of the invention, a non-nutritive sweetener such as saccharin, cyclamate, acetosulfame, aspartame, sucralose or a combination thereof, may be used in place of or in addition to carbohydrate sweeteners. Generally, said non-nutritive sweeteners is added at a level of about 0.03% to about 0.15% by weight, and typically from about 0.06% to about 0.10% by weight of the frozen confection. A freezing point depressant is necessary in the absence of carbohydrate solids to depress the freezing point of the frozen confection. A freezing point depressant may be chosen from the group consisting of sodium chloride, glycerin, propylene glycol and polyhydric alcohols, and is generally employed at a level of about 2% to about 5% of the frozen confection.

Natural and/or artificial flavors (e.g. fruit flavors) may also be added to the hydrocolloid mix at levels as would be obvious to those skilled in the art. Also, natural and/or artificial colors may similarly be employed. Optionally, natural fruit juices such as strawberry, cherry, orange, grape, raspberry, blueberry, apple, watermelon, banana, pineapple, cranberry, blackberry, lemon, grapefruit, lime, coconut, pear, peach, etc. may be incorporated to the frozen confection at levels ranging from about 0.1% to 15% and typically from about 7% to 13% by weight of the final frozen confection.

Souring agents can also be added, depending upon the flavoring agent involved. Any souring agent can be used within the scope of the present invention. Citric acid is an example of a typical souring agent. Malic or Tartaric acid can also be used. The concentration of the souring agent depends again on the flavoring agent(s) and desired taste. Preferably about 0 to about 0.5% by weight souring agent is used.

The compositions of the present invention preferably contain food colorings. These food colorings include, but are not limited to, U.S. Certified Food Colors Red #40, Yellow #6, Blue #1. Although not required, food colorings are included to enhance the aesthetic desirability of the composition. Food coloring also helps consumers associate the color with the flavor. For example, raspberry flavor can be dyed red and grape flavor can be dyed blue. A full recitation of all FD&C and D&C colorants useful in the present invention and their corresponding chemical structures can be found in the Kirk-Othmer Encyclopedia of Chemical Technology, 3rd Edition, in Volume 6, at pages 561-595, herein incorporated by reference.

The frozen products of the present invention preferably do not form gels or gel-like networks. Consequently, the frozen compositions of the present invention are preferably substantially free of gelation catalysts such as potassium or calcium as well as such gel forming stabilizers as guar gum, xanthan gum, locust bean gum, gum aragic, carboxymethyl cellulose, methyl cellulose, hydroxy propyl cellulose, hydroxy propyl methyl cellulose, collagen or mixtures thereof. By “substantially free” is meant that the compositions comprise less than an amount of such gelling ingredients that will adversely affect the dissolution properties of the prevent invention. Generally, this will mean that the present compositions comprise no more than about 0.02%, preferably no more than about 0.01%, of such materials. Without being limited by theory, it is believed that gelation prolongs product dissolution time in the mouth which can, in turn, prolong undesirable mouthfeel and taste. The frozen compositions of the present invention are preferably fast dissolving. By “fast dissolving” is meant that the frozen compositions dissolve in less than about 30 seconds, preferably less than about 15 seconds, more preferably less than about 10 seconds, after placing the frozen composition in the oral cavity.

The above-described compositions are frozen. To freeze the above-described compositions, the compositions are placed in an environment at a temperature from −5° C. to about −20° C. In a preferred embodiment, the compositions are frozen and/or kept frozen in a container selected from the group consisting of plastic bags, paper bags, cardboard boxes, waxed paper, foam container and plastic containers

The following examples further describe and demonstrate preferred embodiments within the scope of the present invention. The examples are given solely for the purposes of illustration and are not to be construed as illustrative of limitations of this invention. Many variations thereof are possible without departing from the invention's spirit and scope.

### EXAMPLES

The following compositions are representative of the present invention.

### Examples I-IV

Using conventional mixing technology, the following frozen ice product compositions of the present invention are formulated from the ingredients listed below poured into a suitable single serving or unit dose package, place in a carton or container with at least one other single serving packaged frozen ice product and, then frozen in a multi-package container or carton. Alternatively, the single serving packaged frozen ice product may be frozen prior to storage in the multi-package container or carton.

| | Ex. I | | | |
|---|---|---|---|---|
| Ingredient | w/w % | Ex. II | Ex. III | Ex. IV |
| Dextromethorphan | 0.03 | 0.03 | 0.03 | 0.03 |
| Hydrobromide | | | | |
| Pectin | 0.60 | 0.60 | 0.60 | 0.60 |
| Propane 1,2-Diol Alginate | | | | 0.2 |
| (Kelcoloid LVF) | | | | |
| Sodium Alginate (Protanal | 0.20 | 0.50 | — | — |
| LF200) | | | | |
| Sodium Alginate (Protanal | — | — | 2.0 | — |
| LF10/60) | | | | |
| High Fructose Corn Syrup | 11.00 | 15.00 | 20.00 | 20.00 |
| Potassium Sorbate | 0.03 | 0.03 | 0.03 | 0.03 |
| Sodium Benzoate | 0.03 | 0.03 | 0.03 | 0.03 |
| Citric Acid Anhydrous | 0.40 | 0.40 | 0.40 | 0.40 |
| Sodium Citrate Dihydrate | 0.32 | 0.32 | 0.32 | 0.32 |
| Sodium Chloride | 0.10 | 0.10 | 0.10 | 0.10 |
| Sucralose | 0.05 | 0.05 | 0.05 | 0.05 |
| Raspberry flavor | 0.135 | 0.135 | 0.135 | — |
| Natural berry flavor | 0.015 | 0.015 | 0.015 | — |
| Grape Flavor | — | — | — | 0.30 |
| Ethyl Maltol | 0.02 | 0.02 | 0.02 | 0.02 |
| Monoammonium | 0.04 | 0.04 | 0.04 | 0.04 |
| Glycyrrhizinate | | | | |
| FD& C Red #40 | 0.016 | 0.016 | 0.016 | 0.004 |
| FD& C Blue #1 | — | — | — | 0.0016 |
| Purified Water | Qs to | Qs to | Qs to | Qs to |
| | 100% | 100% | 100% | 100% |

What is claimed is:

1. A frozen composition, comprising:
a.) an amount of a freezing facilitator consisting essentially of a pharmaceutically acceptable salt or ester of alginic acid such that the viscosity of the composition prior to freezing is no more than 300 cps;
b.) optionally, a safe and effective amount of pharmaceutical active;
c.) optionally, a flavor;
d.) optionally, a sweetener; and
e.) water
wherein the frozen composition contains a solids content of greater than 8% and less than 30% and wherein the frozen compositions are frozen quiescently at a temperature range of from −5° C. to about −20° C.

2. A frozen composition according to claim 1, wherein the ester of alginic acid is a propylene glycol ester of alginic acid.

3. A frozen composition according to claim 1 wherein the solids content is less than 25%.

4. A frozen composition according to claim 1 wherein the solids content is from 9 to 20%.

5. A frozen composition according to claim 1, wherein the viscosity of the composition is from 25 cps to 200 cps.

6. A frozen composition according to claim 5, wherein the viscosity of the composition is from 50 cps to 150 cps.

7. A frozen composition according to claim 1 wherein the alginic acid salt is selected from the group consisting of potassium alginate, sodium alginate and mixtures thereof.

8. A frozen composition according to claim 7 wherein the alginic acid salt is sodium alginate.

9. A frozen composition according to claim 1, further comprising a pharmaceutically active ingredient.

10. A frozen composition according to claim 9, wherein the pharmaceutically active ingredient is selected from the group consisting of antipyretic-analgesic-inflammatory agents, antipsychotic drugs, antianxiety drugs, antidepressants, hypnotic-sedative agents, spasmolytics, gastrointestinal function conditioning agents, antacids, antitussive-expectorants, dental buccal drugs, antihistamines, cardiotonics, antiarrhythmic drugs, diuretics, antihypertensive drugs, vasoconstrictors, coronary vasodilators, peripheral vasodilators, cholagogues, antibiotics, chemotherapeutic drugs, antidiabetic agents, drugs for osteoporosis, skeletal muscle relaxants and mixtures thereof.

11. A frozen composition according to claim 1, further comprising a demulcent.

12. A container for storing frozen compositions, the container comprising quiescently frozen compositions, comprising:
a.) an amount of a freezing facilitator consisting essentially of a pharmaceutically acceptable salt or ester of alginic acid such that the viscosity of the composition prior to freezing is no more than 300 cps;
b.) optionally, a safe and effective amount of pharmaceutical active;
c.) optionally, a flavor;
d.) optionally, a sweetener; and
e.) water
wherein the frozen composition contains a solids content of greater than 8% and less than 30% and wherein the frozen compositions are frozen quiescently and/or maintained in a frozen state at a temperature range of from −5° C. to about −20° C.

13. A container for storing frozen compositions according to claim 12, wherein the container is selected from the group consisting of plastic bags, paper bags, cardboard boxes, waxed paper, foam container and plastic containers.

14. A method of preventing or treating the symptoms of a gastrointestinal disorder by administering to an individual having difficulty swallowing tablets or capsules a safe and effective amount of a composition according to claim 10.

15. A method of preventing or treating the symptoms of a respiratory illness by administering to an individual having difficulty swallowing tablets or capsules a safe and effective amount of a composition according to claim 10.
